SEQUENCE LISTING (7 sequences)

SEQ ID NO: 1 - DNA, 15 bp, Artificial
  FRET probe sequence
gccgctgaagggctt

SEQ ID NO: 2 - DNA, 15 bp, Artificial
  FRET probe sequence
ttccttattgatggt

SEQ ID NO: 3 - DNA, 14 bp, Artificial
  FRET probe sequence
tgaactctgcttaa

SEQ ID NO: 4 - DNA, 15 bp, Artificial
  FRET probe sequence
tgcgtctccatggaa

SEQ ID NO: 5 - DNA, 12 bp, Artificial
  FRET probe sequence
gggtctcaatgg

SEQ ID NO: 6 - DNA, 15 bp, Artificial
  FRET probe sequence
tgcctccccggcgcc

SEQ ID NO: 7 - DNA, 15 bp, Artificial
  FRET probe sequence
tttcccctgggtgat

(19) United States
(12) Patent Application Publication
(10) Pub. No.: US 2016/0362745 A1
(43) Pub. Date: Dec. 15, 2016

(54) Flow Cytometry-Based Detection of Fusion Oncogenes Associated with Hematopoietic Disorders Using Forster Resonance Energy Transfer Probes

(71) Applicant: Wake Forest University Health Sciences, Winston-Salem, NC (US)

(72) Inventors: Shaozhou Ken Tian, Clemmons, NC (US); David Douglas Grier, Winston-Salem, NC (US); Michael Wesley Beaty, Winston-Salem, NC (US); Wei Wang, Winston-Salem, NC (US)

(21) Appl. No.: 15/081,255

(22) Filed: Mar. 25, 2016

Related U.S. Application Data
(60) Provisional application No. 62/138,519, filed on Mar. 26, 2015.

(51) Int. Cl. C12Q 1/68

(52) U.S. Cl. CPC C12Q 1/6886; C12Q 1/6818; C12Q 2600/156; C12Q 2600/158; C12Q 2600/112

(57) ABSTRACT

The present invention relates to methods of detecting chromosomal abnormalities in a biological sample using flow cytometry and nucleic acid probes to assist with the same.

### CROSS-REFERENCE TO RELATED APPLICATIONS

This application claims the benefit of U.S. Provisional Application No, 62/138,519, filed Mar. 26, 2015, the content of which is incorporated by reference herein in its entirety.

### STATEMENT REGARDING ELECTRONIC FILING OF A SEQUENCE LISTING

A Sequence Listing in ASCII text format, submitted under 37 C.F.R. §1.821, entitled 9151-199ST25_{<}txt, 1,692 bytes in size, generated on Mar. 25, 2016 and riled via EFS-Web, is provided in lieu of a paper copy, This Sequence Listing is hereby incorporated by reference into the specification for its disclosures.

### FIELD OF THE INVENTION

The present invention relates to methods of using flow cytometry to detect chromosomal abnormalities, such as cytogenic translocations and fusion genetic products, associated with hematopoietic disorders using Forster resonance energy transfer nucleic acid probes. The present invention further relates to nucleic acid probes that report luminescence in the presence of chromosomal abnormalities, such as cytogenic translocations and fusion genetic products, associated with hematopoietic disorders.

### BACKGROUND OF THE INVENTION

The cytogenetics of hematopoietic disorders is a well-established discipline and an aspect of routine patient care. In the ever-developing fields of genetics and molecular pathology certain chromosomal or gene alterations have led to mutation-specific targeted therapy, Examples such as Imantinib (GLEEVAC) targeting the BCR-ABL gene fusion product (9;22) in chronic myelogenous (or myeloid) leukemia/acute lymphoblastic leukemia (CML/ALL) and all-trans retinoic acid (ATRA) targeting of the PML-RARA gene fusion product (15;17) in acute promyelocytic leukemia (APL) have become the standard of care. In general, clinical suspicion guides genetic testing, but since therapy is targeted at a specific fusion gene and subsequent fusion protein, genetic confirmation is often utilized to initiate therapy. A faster and confirmatory diagnosis can be especially critical in patients with APL since these patients are prone to develop disseminated intravascular coagulation (DIC). DIC is a medical emergency with substantial morbidity and mortality that can be reduced by timely diagnosis and treatment with ATRA. Current methods usually rely essentially on fluorescence in situ hybridization (FISH) and polymerase chain reaction (PCR) methods. Although these methods are generally reliable and reproducible, turn-around times can be substantially long depending on, for example, collection methods, assay technique, or patient load. These techniques can require considerable training, equipment, and technical man-power. In terms of patient care, this can equate to increased length of stay and costs while waiting for test results.

CML is a myeloproliferative neoplasm defined by the presence of a BCR-ABL1 fusion gene. 90-95% of eases have t(9;22)(q34;q11.2), wherein BCR gene on chromosome 22 fuses with regions of the ABL1 gene on chromosome 9, the remainder having variant or cryptic translocations. The BCR-ABL1 gene lesion has been detected by fluorescence in situ hybridization (FISH), RT-PCR or Southern blot analysis. The detection of the fusion is implicated in initiation of therapy of CML using tyrosine kinase inhibitors (TKIs).

Complicated cytogenetics is typically limited to only larger academic settings. Smaller hospitals generally have to send their samples to tertiary labs to have such analysis performed. Even in tertiary hospitals, cytogenetic support is not available 24/7. On the other hand, flow cytometry is normally available by some means at any time of the day. Flow cytometry is a standard method to access and characterize hematopoietic disorders. The flow cytometric techniques have relatively short turn-around times. The techniques generally rely on cell population type size, cytoplasmic complexity, and antigen (protein, enzymes, etc) expressivity to classify the given sample. Many different antigens may be assayed depending on clinical suspicions. The methods all use quantitative fluorescence reporting with specific laser excitation requirements. However, these antigens are typically limited to proteins and specific enzymatic substrates in clinical and research fields. Moreover, genetic materials such as mRNA, miRNA and specific genomic DNA have seldom been assayed using flow cytometry.

As such, there is a need for novel methods, such as the methods as set forth herein that can improve turn-around time and may decrease patient-costs in the detection, characterization and/or subclassification of hematopoietic disorders.

### SUMMARY OF THE INVENTION

Aspects of the present invention provide methods of detecting a chromosomal abnormality in a biological sample comprising contacting the biological sample with at least two probes comprising the nucleotide sequence of SEQ ID NOs:1, 2, 3, 4, 5, 6, and 7, or a sequence having sequence identity with at least 10 contiguous nucleotides thereof, under conditions suitable for binding of the probes to nucleic acid sequences of an oncogenic gene fusion present in the biological sample wherein each probe is reactive to a distinct site upstream and/or downstream of the DNA/RNA fusion site of the oncogenic fusion; and detecting by flow cytometry whether the probes bind to nucleic acid sequences of the oncogenic gene fusion, wherein if the probes both upstream and downstream of the DNA/RNA fusion site bind to nucleic acid sequences of the oncogenic fusion, the presence of the chromosomal abnormality is indicated. In a particular aspect, the nucleic acid sequences of the oncogenic gene fusion to which the probes bind is an mRNA. In another particular aspect, this binding is indicated through Forster resonance energy transfer (FRET).

In some aspects, at least one of the probes is reactive with or hybridizes to nucleic acid sequences upstream of the DNA/RNA fusion site of the oncogenic gene fusion and at least one of the probes is reactive with or hybridizes to nucleic acid sequences downstream of the DNA/RNA fusion site of the oncogenic gene fusion. In further aspects, nucleic acid sequences of the oncogenic gene fusions are BCR-ABL fusion products or are PML-RARA fusion products.

According to some aspects, the probe is labeled with a luminescent agent. In further aspects, the luminescent agent is a fluorescent agent, and in some aspects, the fluorescent agent is a fluorescent dye selected from the group consisting of a cyanine dye, a sulfonated coumarin dye, a sulfonated rhodamine dye, a sulfonated xanthene dye and a sulfonated cyanine dye, or a combination thereof. In a particular aspect of the invention, one luminescent agent acts as a fluorescence donor and a second luminescent agent acts as a fluorescence acceptor.

Aspects of the present invention further provide methods of detecting cancerous cells comprising contacting a biological sample comprising cells with at least two probes comprising the nucleotide sequence of SEQ ID NOs:1, 2, 3, 4, 5, 6, and 7, or a sequence having sequence identity with at least 10 contiguous nucleotides thereof, under conditions suitable for binding of the probes to an oncogenic fusion product present in the biological sample wherein each probe is reactive to a distinct site on the oncogenic fusion product; and detecting by flow cytometry whether the probes bind to the oncogenic fusion product, wherein if the probes bind to the oncogenic fusion product, the presence of cancerous cells is indicated.

Additional aspects of the present invention provide methods of monitoring cancer treatment response comprising contacting a biological sample with at least two fluorescent probes comprising the nucleotide sequence of SEQ ID NOs:1, 2, 3, 4, 5, 6, and 7, or a sequence having sequence identity with at least 10 contiguous nucleotides thereof, under conditions suitable for binding of the fluorescent probes to an oncogenic fusion product present in the biological sample wherein each fluorescent probe is reactive to a distinct site on the oncogenic fusion product; and detecting by flow cytometry whether the fluorescent probes bind to the oncogenic fusion product, and comparing the fluorescence to the fluorescence observed during a previous contacting step with a biological sample, wherein a change in fluorescence indicates the cancer treatment response.

Further aspects of the present invention provide a probe comprising the nucleotide sequence of SEQ ID NOs:2, 3, 4, 5, 6, and 7, or a sequence having sequence identity with at least 10 contiguous nucleotides thereof. In some aspects, the probe is labeled with a luminescent agent, and the luminescent agent can be a fluorescent agent. In some aspects, the fluorescent agent is a fluorescent dye selected from the group consisting of a cyanine dye, a sulfonated coumarin dye, a sulfonated rhodamine dye, a sulfonated xanthene dye and a sulfonated cyanine dye, or a combination thereof.

Aspects of the present invention farther provide a pair of probes, wherein the pair is the nucleotide sequence consisting of the group selected from the following:

SEQ ID NO:1 and SEQ ID NO:2;

SEQ ID NO:1 and SEQ ID NO:3;

SEQ ID NO:1 and SEQ ID NO:4;

SEQ ID NO:5 and SEQ ID NO:6; and

SEQ ID NO:5 and SEQ ID NO:7, or a sequence having sequence identity with at least 10 contiguous nucleotides thereof,

In some aspects, the pair of probes is labeled with a luminescent agent, and the luminescent agent may be a fluorescent agent. In some aspects, the fluorescent agent is a fluorescent dye selected from the group consisting of a cyanine dye, a sulfonated coumarin dye, a sulfonated rhodamine dye, a sulfonated xanthene dye and a sulfonated cyanine dye, or a combination thereof.

Aspects of the invention also provide kits comprising the pair of probes as described herein.

In other aspects of the invention, the detection of nucleic acid sequences coding for an oncogenic gene fusion product are for detecting the presence of hematopoietic disorders. More particular aspects relate to the detection of CML/ALL and/or APL.

in yet other aspects of the invention, the detection of nucleic acid sequences coding for an oncogenic gene fusion product are for determining the likelihood that a subject may develop disseminated intravascular coagulation (DIC).

Methods and probes of the present invention can improve detection, characterization and/or subclassification of hematopoietic disorders using flow cytometry methods, improve turn-around time with gene-directed therapies, and/or decrease medical costs.

These and other aspects of the invention are set forth in more detail in the description of the invention below.

### BRIEF DESCRIPTION OF THE DRAWINGS

FIG. 1 is a depiction of exemplary Forster resonance energy transfer (FRET) probe detection of nucleic acid sequences. When two specific nucleotide sequences are adjacent to one another, FRET probes for the detection of such sequences, containing a donor and acceptor fluorophore, will emit a detectable fluorescence signal.

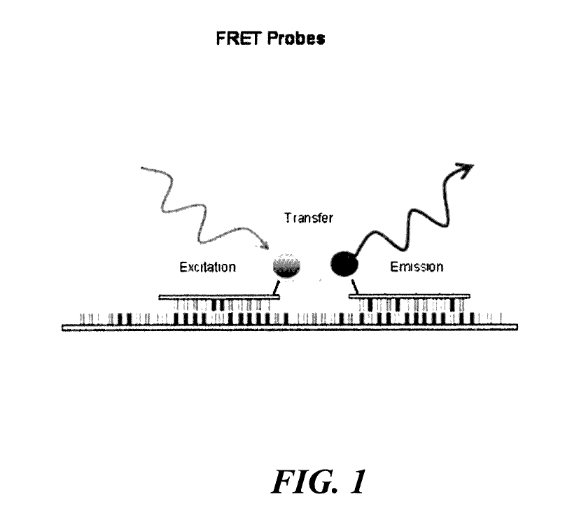

### DETAILED DESCRIPTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description, of the invention herein is for the purpose of describing particular embodiments only and is not intended to be limiting of the invention.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 C.F.R. §1.822 and established usage.

Except as otherwise indicated, standard methods known to those skilled in the art may be used for cloning genes, amplifying and detecting nucleic acids, and the like. Such techniques are known to those skilled in the art, See, e.g., Sambrook et al., Molecular Cloning: A Laboratory Manual 2nd Ed. (Cold Spring Harbor, NY, 1989); Ausubel et al. Current Protocols in Molecular Biology (Green Publishing Associates, Inc. and John Wiley & Sons, Inc., New York).

All publications, patent applications, patents, patent publications and other references cited herein are incorporated by reference in their entireties for the teachings relevant to the sentence and/or paragraph in which the reference is presented.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±20%, ±10%, ±5%, ±1%, ±0.5%, or even ±0.1% of the specified amount.

The transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461, 463 (CCPA 1976) (emphasis in the original); see also MPEP §2111.03.

The term “consists essentially of” (and grammatical variants), as applied to a polynucleotide or polypeptide sequence of this invention, means a polynucleotide or polypeptide that consists of both the recited sequence (e.g., SEQ ID NO and a total of ten or less (e.g., 1, 2, 3, 4, 5, 6, 7, 8, 9, or 10) additional nucleotides or amino acids on the 5′ and/or 3′ or N-terminal and/or C-terminal ends of the recited sequence such that the function of the polynucleotide or polypeptide is not materially altered. The total of ten or less additional nucleotides or amino acids includes the total number of additional nucleotides or amino acids on both ends added together.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The terms “nucleotide sequence” “nucleic acid,” “nucleic acid molecule,” “oligonucleotide” and “polynucleotide” are also used interchangeably herein to refer to a heteropolymer of nucleotides and are presented herein in the 5′ to 3′ direction, from left to right and are represented using the standard code for representing the nucleotide characters as set forth in the U.S. sequence rules, 37 CFR §§1.821-1.825 and the World intellectual Property Organization (WIPO) Standard ST.25. The term polynucleotide, nucleotide sequence, or nucleic acid refers to a chain of nucleotides without regard to length of the chain, unless otherwise specified. It will also be understood that DNA nucleotide sequence, nucleic acid, nucleic acid molecule, oligonocleotide and polynucleotide, may represent a corresponding RNA nucleotide sequence, nucleic acid, nucleic acid molecule, oligonucleotide and polynucleotide, wherein the thymine (T) base in the DNA nucleotide sequence, nucleic acid, nucleic acid molecule, oligonocleotide and polynucleotide is replaced with uracil (U) in the RNA nucleotide sequence, nucleic acid, nucleic acid molecule, oligonocleotide and polynucleotide.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state, “Isolated” does not mean that the preparation is technically pure (homogeneous), but it is sufficiently pure to provide the polypeptide or nucleic acid in a form in which it can be used for the intended purpose.

The term “fragment,” as applied to a polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., 90%, 92%, 94% 95%, 96%, 97%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, any intervening number of nucleotides, or more consecutive nucleotides of a nucleic acid or nucleotide sequence according to the invention.

The term “fragment,” as applied to a polypeptide will be understood to mean an amino acid sequence of reduced length relative to a reference polypeptide or amino acid sequence and comprising, consisting essentially of, and/or consisting of an amino acid sequence of contiguous amino acids identical or almost identical (e.g., 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of and/or consist of peptides having a length of at least about 4, 6, 8, 10, 12, 15, 20, 25, 30, 35, 40, 45, 50, 75, 100, 150, 200, any intervening number of amino acids, or more consecutive amino acids of a polypeptide or amino acid sequence according to the invention.

As used herein, the terms “protein” and “polypeptide” are used interchangeably and encompass both peptides and proteins, unless indicated otherwise.

As used herein, a “functional” polypeptide or “functional fragment” is one that substantially retains at least one biological activity normally associated with that polypeptide (e.g., target protein binding). In particular embodiments, the “functional” polypeptide or “functional fragment” substantially retains all of the activities possessed by the unmodified peptide. By “substantially retains” biological activity, it is meant that the polypeptide retains at least about 20%, 30%, 40%, 50%, 60%, 75%, 85%, 90%, 92%, 94%, 95%, 96%, 97%, 98%, 99%, or more, of the biological activity of the native polypeptide (and can even have a higher level of activity than the native polypeptide). A “non-functional” polypeptide is one that exhibits little or essentially no detectable biological activity normally associated with the polypeptide (e.g., at most, only an insignificant amount, e.g., less than about 10% or even 5%). Biological activities such as protein binding can be measured using assays that are well known in the art and as described herein.

As used herein, a “gene fusion” is when two heterologous nucleotide sequences or fragments thereof coding for two (or more) different polypeptides, or fragments thereof, are fused together in the correct translational, reading frame. A “gene fusion product” includes an RNA, more particularly, an mRNA, produced from the transcription of a gene fusion, as well as a polypeptide or protein resulting from the translation of an RNA, more particularly an mRNA, produced from the transcription of a gene fusion. The two or more different polypeptides, or fragments thereof, coded for by translating an RNA, more particularly an mRNA, gene fusion product include both those not found fused together in nature, and/or include naturally occurring mutants. An “oncogenic gene fusion,”, as used herein, refers to a gene fusion that is associated with causing cancer. In many cases, naturally-occurring oncogenic gene fusions are the result of chromosomal translocations. An “oncogenic gene fusion product,” as used herein, refers to an RNA, more particularly an mRNA, produced from the transcription of a gene fusion that is associated with causing cancer, as well as a fusion protein or product coded for by translating an RNA, more particularly, an mRNA, gene fusion product that is associated with causing cancer.

A “probe” as used herein refers to an isolated oligonucleotide sequence that may hybridize to a target nucleic acid. The probe and the target nucleic acid may either by a DNA or an RNA sequence. In an embodiment, the target nucleic acid is an mRNA, In another embodiment, the target nucleic acid is an amplification product of an mRNA. The probe need not have exact complementarity to the desired target, but should have sufficient complementarity to bind to the region of interest using the methods of the invention. To achieve this generally requires a matching sequence with at least about 80%, 85%, 90% complementarity, preferably about 95%, 96%, 97%, 98%, 99% complementarity, and most preferably about 100% complementarity to the target.

As used herein, “complementary” polynucleotides are those that are capable of hybridizing via base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the ease of RNA: For example, the sequence “A-GT” binds to the complementary sequence “T-C-A” It is understood that two polynucleotides may hybridize to each other even if they are not completely or fully complementary to each other, provided that each has at least one region that is substantially complementary to the other. Polynucleotides that hybridize to each other that are not completely or fully complementary to each other may include degenerate bases, i.e., bases that have multiple possible alternatives as would be understood by one of skill in the art, e.g., W (A or T/U), S (G or C), M (A or C), K (G or T/U), R (A or G), Y (C or T/U), B (C, G, or T/U), D, (A, G or T/U), H (A, C or T/U), V (A, C or G) and N (A, C, G or T).

The term “complementary” includes “substantially complementary” which is intended to refer to a probe which will specifically bind to the region of interest on a chromosome under the test conditions which are employed, and thus be useful for detecting and localizing the region. Complementarity will be extensive enough so that the probes will form specific and stable hybrids with the target nucleic acid under the hybridization conditions used. Persons of ordinary skill in the art will be able to determine suitable sequences through the general knowledge available in the art, and by routine experimentation, using the disclosure provided herein.

A detectable moiety such as a label may be attached to the probe. Exemplary labels include radioactive isotopes, enzyme substrates, co-factors, ligands, luminescent or fluorescent agents, haptens, and enzymes. Methods for labeling and guidance in the choice of labels appropriate for various purposes are discussed, for example, in Sambrook et al., Molecular Cloning: A Laboratory Manual, Cold Spring Harbor Laboratory Press (1989) and Ausubel et at., Current Protocols in Molecular Biology, Greene Publishing Associates and Wiley-Intersciences (1987).

In a particular embodiment, a probe includes at least one fluorophore, such as an acceptor fluorophore or donor fluorophore. For example, a fluorophore can be attached at the 5′- or 3′-end of the probe. In a further embodiment, the probe hybridizes to a nucleotide sequence at which a gene fusion has occurred. Such probes may hybridize to either sequences upstream (5 to the gene fusion) of the gene fusion, or downstream (3′ to the gene fusion) of the gene fusion, In yet a further embodiment, two probes, one to sequences upstream of the gene fusion and one to sequences downstream of the gene fusion, are used, wherein the probe to sequences upstream of the gene fusion has a fluorophore attached to its 3′-end and the probe to sequences downstream of the gene fusion has a fluorophore attached to its 5′-end, in yet another embodiment, the probe to sequences upstream of the gene fusion has a donor fluorophore attached to its 3′-end and the probe to sequences downstream of the gene fusion has an acceptor fluorophore attached to its 5′-end. The nature of the donor and acceptor fluorophores is not particularly limited and may be any which may be available to and appreciated by one of skill in the art. In a particular non-limiting example, the donor fluorophore is ALEXAFLUOR® 488 and the acceptor fluorophore is Cyanine5.5 (CY5,5™). In another non-limiting example, the donor fluorophore. is ALEXAFLUOR® 514 and the acceptor fluorophore is Cyanine5 (CY5™).

As used herein, “detection” refers to determining if an agent (such as a signal or particular nucleotide or amino acid) is present or absent. Detection may involve fluorescence excitation at a light wavelength that is absorbed by a fluorophore. In an embodiment, such a fluorophore may either emit a fluorescence signal that may be detected or measured, or said fluorophore may act as a donor fluorophore in which energy from the fluorescence excitation is transferred to, for example, an acceptor fluorophore, which may emit a detectable or measurable fluorescence signal at a wavelength differing from fluorescence emission wavelength of the donor fluorophore, for example, as in FRET. In some examples, this can further include quantification. For example, use of the disclosed probes in particular examples permits detection of a fluorophore, for example detection of a signal, for example, a fluorescence emission, from the fluorophore or an acceptor fluorophore, which can be used to determine if a nucleic acid corresponding to nucleic acid of an oncogenic fusion product. The method of detection of a signal, such as a fluorescence emission, is not particularly limited. In an embodiment, for example, wherein fluorescence emission is detected, any standard fluorometric detection method may be used as would be understood by one of skill in the art, in a further embodiment, detection may take place in the presence of a fluorescence quencher, which may be used to reduce, for example, background fluorescence and increase signal to noise ratio, in order to enhance detection of the fluorescence signal.

The nature of the fluorescence quencher is not particularly limited, and the quencher may be any that would be appreciated by one of skill in the art. In a particular embodiment, the fluorescence quencher, for example, dabsyl, may be conjugated with one of probes, for example, with the probe containing a donor fluorophore. In a further embodiment, the quencher is displaced from the probe containing the donor fluorophore through, for example, ligation of the probe containing the acceptor fluorophore with the probe containing the donor fluorophore upon hybridization of both probes to the gene fusion, such as in, for example, quenched autoligation-FRET (QFRET).

The term “chromosomal abnormality” or “chromosomal aberration,” which can be used interchangeably, refer to rearrangements, translocations, inversions, insertions, deletions and other mutations within or among chromosomes resulting in the amplification of the expression of a genetic product, the expression of a new genetic product and/or amplification of the expression of a new genetic product. Often, the chromosomal abnormality is involved in the development of malignancies where the new genetic combination can be the foundation of a malignancy.

The term “hematopoietic disorder,” as used herein, refers to any disease, disorder, or condition related to the formation of blood cellular components. Examples of organs or tissues involved in the formation of blood cellular components include, but are not limited to, bone marrow and lymph nodes. Examples of hematopoietic disorders include, without limitation, hereditary, congenital, as well as acquired disorders. Hereditary and congenital disorders include, but are not limited to, bone marrow failure syndromes and primary deficiency syndromes. Acquired disorders include but are not limited to those related to nutritional deficiencies, such as nutritionally related anemias resulting from iron, folate or vitamin B12 deficiencies, infectious processes and neoplastic disorders, for example, cancer.

The term “cancer,” as used herein, refers to any benign or malignant abnormal growth of cells. Examples include, without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is a type of leukemia.

As used herein, the term “subject” refers to humans and other animals. Suitable subjects include mammals such as humans, as well as those mammals of importance due to being endangered, such as Siberian tigers; of economic importance, such as animals raised on farms; animals of social importance to humans, such as animals kept as pets or in zoos; and research animals, such as mice, rabbits, guinea pigs, ferrets, dogs, cats, monkeys, and apes, Examples of such animals include but are not limited to: carnivores such as cats and dogs; swine, including pigs, hogs, and wild boars; ruminants and/or ungulates such as cattle, oxen, sheep, giraffes, deer, goats, bison, and camels; horses; and poultry. The present invention finds use in veterinary and medical applications as well as research applications. In particular embodiments, the subject may be diagnosed with a hematopoietic disorder or suspected to have a hematopoietic disorder.

Genetic materials such as mRNA, miRNA, and specific genomic DNA have seldom been assayed using flow cytometry. The present invention provides, among other things, methods of using flow cytometry targeted at Forster resonance energy transfer (FRET) linked complementary nucleic acid probes to assay for common fusion oncogenes in hematopoietic disorders.

In some embodiments of the present invention, methods of detecting a chromosomal abnormality in a biological sample comprises, consists essentially of or consists of contacting the biological sample with at least two probes comprising, consisting essentially of and/or consisting of the sequences GCCGCTGAAGGGCTT (SEQ ID NO:1), TTCCTTATTGATGGT (SEQ ID NO:2), TGAACTCTGCTTAA (SEQ ID NO:3), TGCGTCTCCATGGAA (SEQ ID NO:4), GGGTCTCAATGG (SEQ ID NO:5), TGCCTCCCCGGCGCC (SEQ ID NO:6) and/or TTTCCCCTGGGTGAT (SEQ ID NO:7), or a sequence having sequence identity with at least 10 contiguous nucleotides thereof, under conditions suitable for binding or hybridization of the probes for an oncogenic gene fusion to an oncogenic gene fusion or oncogenic gene fusion product present in the biological sample wherein each probe is reactive to a distinct site or sequence on the oncogenic gene fusion or oncogenic gene fusion product; and detecting by flow cytometry whether the probes bind or hybridize to the oncogenic gene fusion or oncogenic fusion product, wherein if the probes bind or hybridize to the oncogenic gene fusion or oncogenic gene fusion product, the presence of the chromosomal abnormality is indicated. In an embodiment, the probe or probes bind to or hybridize to an mRNA transcribed from an oncogenic gene fusion. In a further embodiment, at least one of the probes hybridizes with nucleic acid sequences upstream of the DNA/RNA fusion site of the oncogenic gene fusion and at least one of the probes hybridizes with nucleic acid sequences downstream of the oncogenic gene fusion.

In particular embodiments, the probe is labeled with a luminescent agent. In particular embodiments, the luminescent agent is a fluorescent agent. In further embodiments, the fluorescent agent is a fluorescent dye selected from the group consisting of a cyanine dye, a sulfonated coumarin dye, a sulfonated rhodamine dye, a sulfonated xanthene dye and a sulfonated cyanine dye, or a combination thereof. According to further embodiments, dyes compatible with this technology include, but are not limited to, ALEXAFLUOR® dyes such as ALEXAFLUOR® 350, 405, 430, 488, 514, 532, 546, 555, 568, 594, 633, 635, 647, 660, 680, 700, 750, and 790, or Cyanine dyes such as Cy2, Cy3, Cy3B, Cy3.5, Cy5, Cy5,5 and Cy7.

In some embodiments, the fluorescent dye is selected from the group consisting of Alexa 350, 405, 430, 488, 514, 532, 546, 555, 568, 594, 633, 635, 647, 660, 680, 700, 750, and 790 and Cy2, Cy3, Cy3B, Cy3.5, Cy5, Cy5.5 and Cy7, or a combination thereof. In some embodiments, the fluorescent dye is ALEXAFLUOR® 488 or Cy5.5, or a combination thereof In other embodiments, the fluorescent dye is ALEXAFLUOR® 514 and Cy5, or a combination thereof.

According to embodiments of the present invention, the at least two paired probes can report fluorescence in conditions of fusion transcript production where the probes will also be designed to the specifications of the flow cytometer excitation and fluorescence parameters. In an embodiment, one of the paired probes acts as an acceptor fluorophore and one of the probes acts as a donor fluorophore, wherein fluorescence excitation targets the donor fluorophore.

In particular embodiments, the chromosomal abnormality is a translocation or oncogene fusion protein associated with a hematologic disorder. The hematologic disorder can be hereditary, congenital or acquired. The hematologic disorder can be, for example, a cancer, such as leukemia or lymphoma. In some embodiments, the hematologic disorder is leukemia. In further embodiments, the leukemia is acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL) or large granular lymphocytic leukemia.

In some embodiments, the oncogenic gene fusion product detected is the result of a BCR-ABL gene fusion product or the result of a PML-RARA gene fusion product. The gene fusion product may either be a transcription product of a gene fusion-RNA, more particularly miRNA-or a translation product of a gene fusion. In a further embodiment, the oncogenic gene fusion product detected is an mRNA transcribed from a BCR-ABL oncogenic gene fusion. In yet another embodiment, the oncogenic gene fusion product detected is an mRNA transcribed from a PML-RARA oncogenic gene fusion,

The biological sample employed in the embodiments of the present invention can include, but are not limited to, a blood sample, a serum sample, a cell sample, a tissue sample a bone marrow sample, a cell line, for example, the APL cell line NB4, a primary cell line and xenografts. In particular embodiments, the biological sample is a hematological sample. In particular embodiments, the sample is fresh blood obtained and/or utilized within 5, 10, 15, 20, 25, 30, 35, 40, 45, 50, 55, or 60 min or 1, 2, 3, 4, 5, 6, 7, 8, 9, 10, 11, 12, 13, 14, 15, 16, 17, 18, 19, 20, 21, 22, 23 or 24 hours.

Embodiments of the present invention further provide methods of detecting malignant or cancerous cells, the methods comprise, consist essentially of, or consist of contacting a biological sample comprising cells with at least two probes comprising the nucleotide sequence of SEQ NOs:1, 2, 3, 4, 5, 6, and 7, or a sequence having sequence identity with at least 10 contiguous nucleotides thereof, under conditions suitable for binding of the probes to an oncogenic fusion product present in the biological sample wherein each probe is reactive to a distinct site on the oncogenic fusion product; and detecting by flow cytometry whether the probes bind to the oncogenic fusion product, wherein if the probes bind to the oncogenic fusion product, the presence of malignant or cancerous cells is indicated. In particular embodiments, the biological sample includes hematopoietic cells. In particular embodiments, introduction of the probes to the hematopoietic cells does not involve re-sealing the cells. In some embodiments, the hematopoietic cells are hematocytes. The hematocytes include erythrocytes, leukocytes and thrombocytes. In particular embodiments, the cells of interest are leukocytes and hematopoietic stem cells.

Embodiments of the present invention also provide methods of monitoring cancer treatment response comprising, consisting essentially of or consisting of contacting a biological sample from a subject with at least two fluorescent probes comprising the nucleotide sequence of SEQ ID NOs:1, 2, 3, 4, 5, 6, and 7, or a sequence having sequence identity with at least 10 contiguous nucleotides thereof, under conditions suitable for binding of the fluorescent probes to an oncogenic fusion product present in the biological sample wherein each fluorescent probe is reactive to a distinct site on the oncogenic fusion product; and detecting by flow cytometry whether the fluorescent probes bind to the oncogenic fusion product, and comparing the fluorescence to the fluorescence observed during a prior contacting step with a different biological sample from the subject, wherein a change in fluorescence indicates the cancer treatment response. For example, a decrease or absence of fluorescence observed after cancer treatment with an agent compared to fluorescence observed prior to treatment with the agent or a different agent can indicate patient responsiveness to treatment and/or treatment efficacy. In a further embodiment, the method of the invention may be used to monitor remission of disease following patient treatment, where a decrease or the absence of observed fluorescence as compared to fluorescence observed prior to treatment is indicative of remission. Thus, in some embodiments, a biological sample is collected after commencement of cancer treatment, either initial treatment or after initiating a new round of treatment. In some embodiments, a biological sample from a subject is obtained prior to commencement of cancer treatment, either before initial treatment or before initiating a new round of treatment, or is obtained earlier in the cancer treatment than a biological sample of comparison.

In some embodiments, the fluorescence is provided by a fluorescent dye as described above.

Embodiments of the present invention also provide a probe set or pair of probes, wherein the pair is the nucleotide sequence comprising, consisting essentially of or consisting of SEQ ID NO:1 and SEQ ID NO:2; SEQ ID NO; I and SEQ ID NO:3; SEQ ID NO:1 and SEQ ID NO:4; SEQ ID NO:5 and SEQ ID NO:6; and SEQ ID NO:5 and SEQ ID NO:7, or a sequence having sequence identity with at least 10 contiguous nucleotides thereof in some embodiments, a cocktail of probes is provided including more than one probe set or pair of probes. In some embodiments, the probes are labeled with a detectable moiety. The detectable moiety can be a luminescent agent. The luminescent agent can be a fluorescent agent as described above.

Embodiments of the present invention further provide kits comprising, consisting essentially of or consisting of pair of probes of SEQ ID NO:1 and SEQ ID NO:2; SEQ ID NO:1 and SEQ ID NO:3; SEQ ID NO:1 and SEQ ID NO:4; SEQ ID NO:5 and SEQ ID NO:6; and SEQ ID NO:5 and SEQ ID NO:7, or a sequence having sequence identity with at least 10 contiguous nucleotides thereof.

The kits further include the elements necessary to carry out the processes described above. Such a kit may comprise a carrier being compartmentalized to receive in close confinement therein one or more containers, such as tubes or vials. One of the containers may contain unlabeled or detectably labeled probes. The labeled probes may be present in lyophilized form or in an appropriate buffer as necessary. One or more containers may contain, one or more enzymes or reagents to be utilized in desired reactions. These enzymes may be present by themselves or in admixtures, in lyophilized form or in appropriate buffers. The kit may contain all of the additional elements necessary to carry out techniques of the invention, such as buffers, extraction reagents, fixation agents, permeability agents, enzymes, pipettes, plates, nucleic acids, nucleoside triphosphates, filter paper, gel materials, transfer materials, autoradiography supplies, instructions and the like.

The present invention is more particularly described in the following examples that are intended as illustrative only since numerous modifications and variations therein will be apparent to those skilled in the art.

### EXAMPLE 1

### Experimental Methods

Sample Preparation for Flow Cytometry. The oligonucleotides are diluted to a final working concentration of 100 nM each in the final buffer. The buffer consists of 1×PBS with 10 of BSA and Salmon sperm DNA. There are at least two probes per reaction depending on alternative translocations. Sample preparation for flow cytometry is exemplified as follows using the IntraPrep™ protocol:
1. 50 μL of sample (blood, BM, cell, etc.) into a flow cytometry tube
2. 100 μL of reagent 1
3. incubate 15 minutes
4. Wash with PBS
5. 100 μL of reagent 2
6. Incubate 5 minutes
7. Add 20 μL of probes
8. incubate 15 minutes
9. Wash
10. Run on flow cytometer (cells may optionally be sealed).

### EXAMPLE 2

### Detection of BRC-ABL Chromosomal Translocation

Detection of BCR-ABL mRNA using protocol of the invention. Translocation junction sequence specific reporter probes for the BCR-ABL gene fusion were used. The probes were BCR1 p210 (e13, e14), BCR1 p190 and ABL1. Flow cytometry as outlined in Example 1 was used to examine a number of cases. The results are as outlined below:
Method has been tried on 40 cases
35 peripheral blood and 5 bone marrows
10 positive cases (3 BM)
Confirmed positive by FISH and/or PCR
No false positives
Eosinophils have autofluorescence
25 Negative cases
Confirmed negative by FISH or PCR
False negatives pending further analysis in low burden disease
   All results were correlated with cytogenetics in diagnosis and/or PCR in clinical monitored patients on TKIs.

### EXAMPLE 3

### Detection of PML-RARA Chromosomal Translocation

Detection of PML-RARA mRNA using protocol of the invention. Translocation junction sequence specific reporter probes for the PML-RARA gene fusion are used. The flow cytometry protocol and chromosomal translocation detection method used is similar to the flow cytometry protocol outlined in Example 1 and the chromosomal detection method outlined in Examiner 2. Positive and negative cases are confirmed by FISH and/or PCR and all results are correlated with cytogenetics in diagnosis and/or PCT in clinically monitored patients on TKIs.

The foregoing is illustrative of the present invention, and is not to be construed as limiting thereof. The invention is defined by the following claims, with equivalents of the claims to be included therein.

That which is claimed is:

1. A method of detecting a chromosomal abnormality in a biological sample comprising:
contacting the biological sample with at least two probes comprising the nucleotide sequence of SEQ ID NOs:1, 2, 3, 4, 5, 6, and 7, or a sequence having sequence identity with at least 10 contiguous nucleotides thereof, under conditions suitable for binding or hybridizing of the probes to an oncogenic gene fusion or an oncogenic gene fusion product present in the biological sample wherein each probe binds or hybridizes to a distinct site on the oncogenic gene fusion or an oncogenic gene fusion product; and
detecting by flow cytometry whether the probes bind or hybridize to the oncogenic gene fusion or oncogenic gene fusion product, wherein if the probes bind or hybridize to the oncogenic gene fusion or oncogenic gene fusion product, the presence of the chromosomal abnormality is indicated.

2. The method of claim 1, wherein the oncogenic gene fusion product is an RNA or an mRNA transcribed from an oncogenic gene fusion.

3. The method of claim 1, wherein at least one of the probes binds or hybridizes with sequences upstream of the oncogenic gene fusion in the oncogenic gene fusion product and at least one of the probes binds or hybridizes with sequences downstream of the oncogenic gene fusion in the oncogenic gene fusion product.

4. The method of claim 1, wherein the chromosomal abnormality is a translocation or oncogene fusion protein associated with a hematopoietic disorder.

5. The method of claim 4, wherein the hematopoietic disorder is a cancer.

6. The method of claim 4, wherein the hematopoietic disorder is leukemia.

7. The method of claim 6, wherein the leukemia is acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL) or large granular lymphocytic leukemia.

8. The method of claim 1, wherein the probe is labeled with a luminescent agent.

9. The method of claim 8, wherein the luminescent agent is a fluorescent agent.

10. The method of claim 9, wherein the fluorescent agent is a fluorescent dye selected from the group consisting of a cyanine dye, a sulfonated coumarin dye, a sulfonated rhodamine dye, a sulfonated xanthene dye and a sulfonated cyanine dye, or a combination thereof.

11. The method of claim 10, wherein the fluorescent dye is selected from the group consisting of ALEXAFLUOR® 350, 405, 430, 488, 514, 532, 546, 555, 568, 594, 633, 635, 647, 660, 680, 700. 750, and 790 and CY2™, CY3™, CY3B™, CY5™, CY5.5™ and CY7™, or a combination thereof.

12. A method of detecting cancerous cells comprising:
contacting a biological sample comprising cells with at least two probes comprising the nucleotide sequence of SEQ ID NOs:1, 2, 3, 4, 5, 6, and 7, or a sequence having sequence identity with at least 10 contiguous nucleotides thereof, under conditions suitable for binding or hybridizing of the probes to an oncogenic gene fusion or gene fusion product present in the biological sample wherein each probe is reactive to a distinct site on the oncogenic gene fusion or gene fusion product; and
detecting by flow cytometry whether the probes bind or hybridize to the oncogenic gene fusion or gene fusion product, wherein if the probes bind or hybridize to the oncogenic gene fusion or gene fusion product, the presence of cancerous cells is indicated.

13. A method of monitoring cancer treatment response comprising:
contacting a first biological sample from a subject with at least two fluorescent probes comprising the nucleotide sequence of SEQ NOs:1, 2, 3, 4, 5, 6, and 7, or a sequence baying sequence identity with at least 10 contiguous nucleotides thereof, under conditions suitable for binding or hybridizing of the fluorescent probes to an oncogenic gene fusion or gene fusion product present in the biological sample wherein each fluorescent probe binds or hybridizes to a distinct site on the oncogenic gene fusion or gene fusion product; and
detecting by flow cytometry whether the fluorescent probes bind or hybridize to the oncogenic gene fusion or gene fusion product, and comparing the fluorescence to the fluorescence observed during a prior contacting step with a second biological sample from the subject, wherein a change in fluorescence indicates the cancer treatment response.

14. The method of claim 13, wherein the first biological sample is collected after commencement of cancer treatment.

15. The method of claim 13, wherein the second biological sample from the subject is obtained prior to commencement of cancer treatment, or is obtained earlier in the cancer treatment than the first biological sample.

16. A pair of probes, wherein the pair is the nucleotide sequence consisting of the group selected from the following:
(a) SEQ ID NO:1 and SEQ NO:2;
(b) SEQ ID NO:1 and SEQ NO:3;
(c) SEQ ID NO:1 and SEQ ID NO:4;
(d) SEQ ID NO:5 and SEQ ID NO:6; and
(e) SEQ ID NO:5 and SEQ ID NO:7, or a sequence having sequence identity with at least 10 contiguous nucleotides thereof.

17. The pair of probes of claim 16, wherein the pair is labeled with a luminescent agent.

18. The pair of probes of claim 17, wherein the luminescent agent is a fluorescent agent.

19. The pair of probes of claim 18, wherein the fluorescent agent is a fluorescent dye selected from the group consisting of a cyanine dye, a sulfonated coumarin dye, a sulfonated .rhodamine dye, a sulfonated xanthene dye and a sulfonated cyanine dye, or a combination thereof.

20. The pair of probes of claim 19, wherein the fluorescent dye is selected from the group consisting of ALEXAFLUOR® 350, 405, 430, 488, 514, 532, 546, 555, 568, 594, 633, 635, 647, 660, 680, 700, 750, and 790 and CY2™, CY3™, CY3B™, CY5™, CY5.5™ and CY7™, or a combination thereof.
